(19) United States
(12) Patent Application Publication
(10) Pub. No.: US 2021/0059658 A1
(43) Pub. Date: Mar. 4, 2021

(54) SURGICAL PORT ASSEMBLIES AND METHODS

(71) Applicant: JOHN P. CONNORS, III, Atlanta, GA (US)

(72) Inventor: JOHN P. CONNORS, III, Atlanta, GA (US)

(21) Appl. No.: 16/552,184

(22) Filed: Aug. 27, 2019

(51) Int. Cl. A61B 17/02

(52) U.S. Cl. CPC A61B 17/0218; A61B 2017/0212

(57) ABSTRACT

A surgical port for protecting nearby tissue is described. In one example, the port includes a base portion connected to a hollow tubular portion having a beveled end. The base portion is sized and shaped to protect the surrounding tissue from heat and friction. The base portion may include one or more holes for receiving sutures to releasably secure the surgical port to the body during use. The beveled end of the tubular portion facilitates lateral movement of surgical instruments toward the short side of the bevel. The base portion may include an indicator such as a notch that provides a visual and tactile cue to the surgeon about the orientation of the beveled end. Using the indicator as a guide, the surgeon can release and rotate the surgical port to another orientation without removing it from the body and re-inserting it.

### TECHNICAL FIELD

The disclosed subject matter includes examples of surgical ports and, in particular, surgical port assemblies and methods related to invasive surgical procedures.

### BACKGROUND

A cannula is a thin, hollow tube that can be inserted into the body through a small incision. A suction cannula is used for the removal of fluids or other substances from the body, such as fluids or fatty tissue, using a suction pump. Liposuction, for example, typically involves the insertion of a suction cannula through a small incision into an area of unwanted fat. The suction cannula can be manipulated to break apart or disrupt the fatty tissues, which are then removed from the area.

Laser-assisted liposuction uses a specialized suction cannula that uses laser energy to heat and liquefy the fatty tissue, making it easier to remove by suction. Ultrasound can also be used in assisted liposuction procedures.

Manipulation of a cannula during a procedure can injure one or more layers of the skin, especially around the incision. In laser-assisted procedures, the cannula can become hot and injure or burn the skin around the incision.

### BRIEF DESCRIPTION OF THE DRAWINGS

Features of the various implementations disclosed will be readily understood from the following detailed description, in which reference is made to the appending drawing figures. A reference numeral is used with each element in the description and throughout the several views of the drawing. When a plurality of similar elements is present, a single reference numeral may be assigned to like elements, with an added lower-case letter referring to a specific element.

The various elements shown in the figures are not drawn to-scale unless otherwise indicated. The dimensions of the various elements may be enlarged or reduced in the interest of clarity. The several figures depict one or more implementations and are presented by way of example only and should not be construed as limiting. Included in the drawings are the following figures:

FIG. 1 is a sectional view of a surgical port, in accordance with some implementations.

FIG. 2 is a perspective view of a surgical port with holes in the base portion, in accordance with some implementations.

FIG. 3 is an illustration of a surgical instrument inserted into and through a surgical port, in accordance with some implementations.

### DETAILED DESCRIPTION

The following detailed description includes numerous details and examples that are intended to provide a thorough understanding of the subject matter and its relevant teachings. Those skilled in the relevant art may understand how to apply the relevant teachings without such details. This disclosure is not limited to the specific devices, systems, and methods described because the relevant teachings can be applied or practiced in a variety of ways. The terminology and nomenclature used herein is for the purpose of describing particular aspects only and is not intended to be limiting.

Those skilled in the relevant art will recognize and appreciate that many changes can be made to the various aspects of the implementations described herein, while still obtaining the beneficial results. It will also be apparent that some of the desired benefits can be obtained by selecting some of the features, but not others. Accordingly, those who work in the art will recognize that many modifications and adaptations are possible and may even be desirable in certain applications, and that these are part of the disclosure.

The terms “comprising” and “including,” and any forms thereof, are intended to indicate a non-exclusive inclusion; that is, to encompass a list that includes the items listed and may include others not expressly listed. As used herein, the singular forms “a,” “an” and “the” include plural referents unless the context clearly dictates otherwise. Thus, for example, reference to a component can include two or more such components unless the context indicates otherwise. Relational terms such as “first” and “second” and “subsequent” and the like may be used solely to distinguish one element or action from another, without implying any particular order between such elements or actions.

The terms “optional” or “optionally” mean that the subsequently described element or action may or may not occur. In other words, such a description includes instances where the element or action occurs and instances where it does not.

Then term “facilitate” means to aid, assist, enable, improve, or make easier. The term “inhibit” means to hinder, impede, restrain, thwart, oppose, or make more difficult.

The term “indicia” refers to any combination of letters, words, text, numbers, symbols, diagrams, pictures, photographs, trademarks, and graphics, in any colors.

The words “proximal” and “distal” are used to describe items or portions of items that are situated closer to and away from, respectively, a user or a viewer. Thus, for example, the near end or other portion of an item may be referred to as the proximal end, whereas the generally opposing portion or far end may be referred to as the distal end.

Manipulation of a cannula or other surgical instrument during a procedure can injure one or more layers of the skin, especially around the incision. In laser-assisted procedures, the cannula can become hot and injure or burn the skin around the incision. There is a need in the art for a surgical port that protects the skin from injury without inhibiting the mobility or utility of the surgical instrument.

Although the various embodiments and implementations are described with reference to a surgical port for use in liposuction procedures, the systems and methods described herein may be applied to and used with any of a variety of procedures, especially those in which protection is desired for the tissue or other material in and around a port or portal. The term “port” as used herein is generally understood to be a shortened form of the word portal, which refers to an opening, entrance, or gateway.

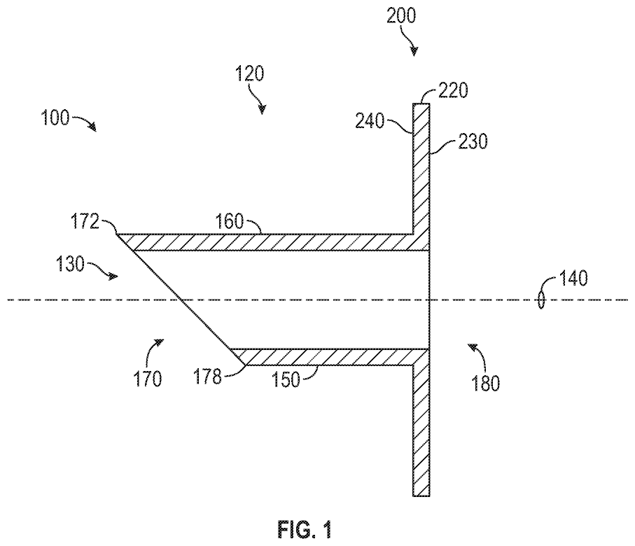

FIG. 1 is a sectional view of a surgical port 100 that includes a tubular portion 120 and a base portion 200. According to some implementations, the tubular portion 120 is hollow and defines a central cavity or channel called a lumen 130 extending through it, along a central longitudinal axis 140. As shown, the lumen 130 extends through the entire port 100 from the distal end 170 to the proximal end 180. The lumen 130 may have a substantially uniform diameter along its entire length.

The tubular portion 120, at the distal end 170, is beveled at an oblique angle with respect to the longitudinal axis 140 to form a bevel tip 172. The inclined surface or bevel, as shown, extends from the bevel tip 172 to a bevel heel 178.

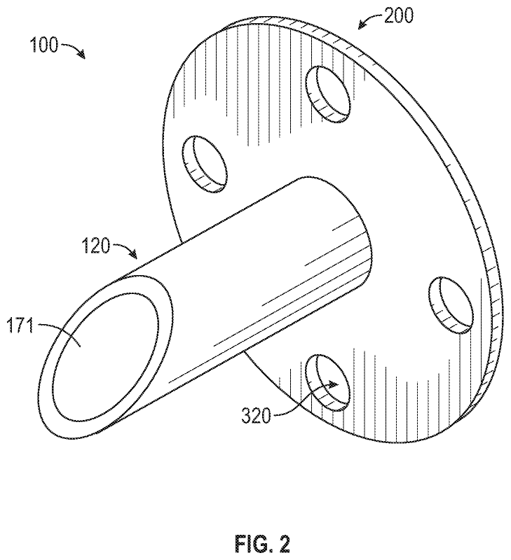

Due to the bevel, the tubular portion 120 has a long side 160 and a short side 150. The long side 160 extends from the bevel tip 172 to the base portion 200; the short side 150 extends from the bevel heel 178 to the base portion 200. In this aspect, the tubular portion 120 takes the form of a hollow cylinder, truncated at an angle, as shown also in FIG. 2.

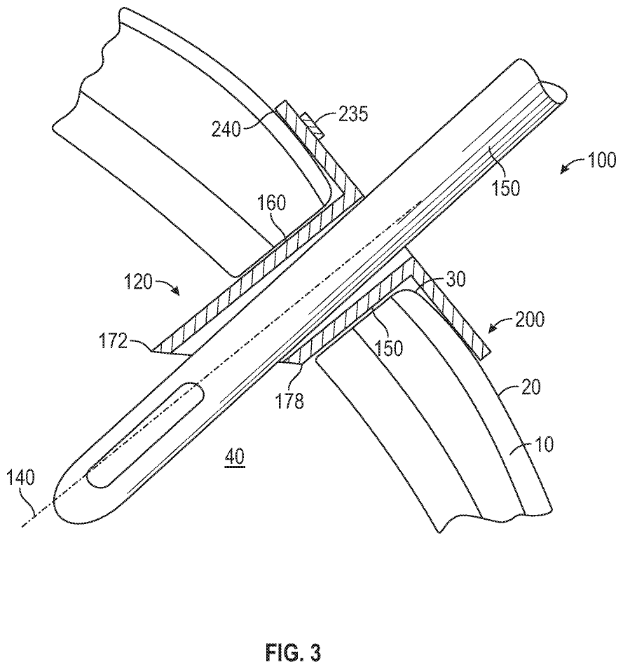

The oblique angle of the bevel, relative to the longitudinal axis 140, may be approximately forty-five degrees, as shown in FIG. 1. The oblique angle is selected to facilitate insertion of the tubular portion 120 into an incision or other opening. For example, as shown in FIG. 3, the tubular portion 120 (starting with the bevel tip 172) has been inserted into an incision 30, the through the skin 10, and into a body cavity 40. During insertion, the bevel on the distal end 170 of the port 100 pushes apart the sides of an opening or incision 30 as the tubular portion 120 is inserted through the skin 10. In this aspect, the beveled shape of the tubular portion 120 facilitates the insertion if the tubular portion 120 into the incision 30.

The base portion 200 is connected to the tubular portion 120 and extends outwardly along the proximal end 180 of the port 100 to a base edge 220. The base portion 200 may be oriented perpendicular to the longitudinal axis 140 of the tubular portion 120, or at some other angle depending on the application or intended use. The base portion 200 is sized and shaped to protect the outer surface 20 of the tissue near the incision 30 from injury during a surgical procedure which may be caused by the heat of a surgical instrument or by physical forces (friction caused by movement, especially repeated manipulation of an instrument). The base portion 200 in some embodiments may be about 19 millimeters in diameter and about 1.5 millimeters in thickness.

The base portion 200 is also sized and shaped to inhibit the insertion of the base portion 120 into the incision 30, while allowing the tubular portion 120 to remain inserted. The base portion 200 is also sized and shaped to facilitate grasping by a human hand, especially the fingers. A generally circular base portion 200 may be referred to as an annular flange extending radially to a perimeter edge. The base portion 200 has a distal surface 240 and generally opposing proximal surface 230. As shown in FIG. 3, the distal surface 240 faces and may lie against the outer surface 20 of the tissue, whereas the proximal surface 230 faces the operator or surgeon.

The surgical port 100 may be made of medical-grade plastic, including any of the polycarbonates approved for medical applications. The material may be selected so that the surgical port 100 can withstand the heat of an autoclave or other sterilization process and can be used again. The surgical port 100 may be made using injection molding, extrusion, and any other fabrication techniques that are useful in obtaining the desired size, shape, and characteristics. The surgical port 100 may be constructed so that the connection between the base portion 200 and the tubular portion 120 is substantially rigid or, alternatively, semi-rigid or flexible so that the two portions 120, 200 can bend relative to the other.

The surgical port 100 may be sized to accommodate an instrument or cannula of a particular size. For example, when using a cannula having a diameter of 3 millimeters, the tubular portion 120 may have an outer diameter equal to about 6 millimeters, a short side 150 equal to about 6 millimeters, a long side 160 equal to about 12 millimeters, and a lumen 130 with an interior diameter equal to about 4.5 millimeters. The base portion 200 may be about 19 millimeters in diameter and have a thickness of about 1.5 millimeters.

FIG. 2 is a perspective view of a surgical port 100 that includes a plurality of holes 320 in the base portion 200. As shown, the distal end 170 defines a generally elliptical distal opening 171. The proximal end 180, as shown in FIG. 1, defines a generally circular proximal opening that is oriented along a plane generally perpendicular to the longitudinal axis 140.

The surgical port 100 in some implementations includes a fastening means for releasably securing the base portion 200 to an outer surface 20, such as the skin 10 around an incision 30 (see FIG. 3). The fastening means is described as releasable because the base portion 200 is secured temporarily. The fastening means, in some implementations, inhibits rotation of the base portion 200 and the port 100, as well as securing the port 100 in one or more linear directions.

The fastening means, according to one embodiment, includes a plurality of circumferentially spaced holes 320 in and through the base portion 200, as shown in FIG. 2, together with one or more fasteners, such as suture thread, medical staples, or clips. In use, sutures may be passed through the holes 320 and through the skin 10, at any selected depth, in order to releasably secure the surgical port 100 to the skin 10. Any of a variety of suitable suture materials, stitching patterns, and techniques may be used.

The fastening means may also include a releasable adhesive between the outer surface 20 of the skin and the distal surface 240 of the base portion 200. The distal surface 240 may include no adhesive material whatsoever. In some implementations, the distal surface 240 may also be shaped and textured to facilitate an adherent engagement with the outer surface 20 of the skin. For example, the distal surface 240 may have a texture that is smooth, ridged, pitted, dotted, or otherwise finished to promote or facilitate an adhesive-like attachment to the outer surface 20 of the skin. Likewise, the outer surface 20 of the skin may also be abraded, or polished, or otherwise prepared to facilitate attachment of the distal surface 240 of the base portion 200.

FIG. 3 is an illustration of a surgical instrument 150 inserted into and through a surgical port 100 into a body cavity 40. The base portion 200 is sized and shaped to protect the outer surface 20 of the skin 10 around an incision 30. The surgical port 100 may include a fastening means, as described herein, between the distal surface 240 of the base portion 200 and the outer surface 20 of the skin.

The surgical instrument 150, as shown, is smaller in diameter than the lumen 130 defined by the tubular portion 120 and is oriented in a lateral position; that is, at an acute angle relative to the longitudinal axis 140 of the port 100. The instrument 150, as shown, is positioned in a lateral (and radial) direction toward the bevel heel 178—on the short side 150 of the tubular portion 120. This positioning illustrates how the beveled shape of the tubular portion 120 of the surgical port 100 facilitates lateral movement of a surgical instrument 150, especially toward to the short side 150. By comparison, a truncated hollow cylinder without a beveled shape would not facilitate such lateral movement; in fact, a plain cylinder would inhibit lateral movement.

The surgical port 100, according to some implementations, includes an indicator 235 on the proximal surface 230 of the base portion 200 where the surgeon can see it and/or feel it. As shown, the indicator 235 may be positioned to align longitudinally with the bevel tip 172 of the port 100, thus providing a cue to the surgeon about the orientation of the tubular portion 120 inside the body. In other implementations, the indicator 235 may be positioned to align with the bevel heel 178. The indicator 235 may be a notch or a groove, either on the proximal surface 230 and/or along the edge 220 of the base portion 200. The indicator 235 may be a raised dot or other protuberance, which provides a tactile as well as a visual cue. The indicator 235 may be a colored dot, line, or other indicia having little or no thickness.

In use, the surgeon can use the indicator 235 as a guide when directing the surgical instrument 150 either toward or away from the short side 150. In some procedures, the surgeon may elect to release the surgical port 100 and rotate the port to a new position without removing the tubular portion 120 entirely from the incision 30. The surgeon during this maneuver can use the indicator 235 as a guide for determining the position of the short side 150 without seeing it. In this aspect, the indicator 125 reduces the number of insertions and withdrawals of the port 100 during a procedure and, thus, reduces the risk of injury to the site of the incision 30.

A surgeon can use the surgical port 100 described herein to protect the tissue 10 near an incision 30 from injury during a surgical operation. The surgeon may select a surgical port 100 having a size and shape that will facilitate the insertion and movements of the surgical instruments to be used in an operation. When using a 3-millimeter cannula, as described in the example above, the surgeon may select a surgical port 100 having a 4.5-millimeter lumen 130.

After making an incision 30, the surgeon may insert the hollow tubular portion 120 into the incision 30 as illustrated in FIG. 3. During insertion, the bevel on the distal end 170 of the port 100 pushes apart the sides of the incision 30. In this aspect, the beveled shape of the tubular portion 120 facilitates the insertion if the tubular portion 120 into the incision 30. By viewing the long side 160 and the short side 150 of the tubular portion 120 before insertion, the surgeon may position the tubular portion 120 so that the short side 150 after insertion will be oriented toward an area of concern within the body cavity 40. As shown in FIG. 3, the beveled shape of the tubular portion 120 facilitates lateral movement of a surgical instrument 150 toward the short side 150.

The surgeon may then releasably secure the base portion 200 to the tissue 10 using any of a variety of fastening means described herein. The base portion 200 in one embodiment may include one or more holes 320, as shown in FIG. 2, for receiving one or more sutures to secure the base portion 200 to the tissue 10.

The surgeon may select a surgical port 100 that includes an indicator 235 located on the visible, proximal surface 240 of the base portion 200, as shown in FIG. 3. The indicator 235, as shown, may be positioned to align with the bevel tip 172 on the long side 160, thus providing a cue to the surgeon about the orientation of the tubular portion 120 inside the body. The indicator 235 may be a notch in the edge 220 of the base portion 200, a raised dot or other protuberance, or a colored dot or other indicia. Using the indicator 235 as a reference for the position of the long side 160 of the tubular portion 120, the surgeon may orient the opposing or short side 150 of the tubular portion 120 toward an area of concern within the body cavity 40. This orientation facilitates lateral movement of a surgical instrument 150, relative to the longitudinal axis 140, as illustrated in FIG. 3. In this aspect, the indicator 235 helps orient the surgical port 100 toward the area of concern. After achieving the desired orientation, the surgeon may releasably secure the base portion 200 to the tissue 10 to prevent unintended rotation of the port 100 during use. The surgical instrument 150 can then be manipulated longitudinally (in and out of the lumen 130) and laterally (side to side, relative to the central, longitudinal axis 140), thus reaching the area of concern near the short side 150.

The surgeon may identify one or subsequent areas of concern, where treatment is desired, within reach of the same incision 30. To rotate and re-orient the surgical port 100, the surgeon may release the base portion 200 from the tissue 10; for example, by removing the sutures. The surgeon may then rotate the port 100—without removing the tubular portion 200 from the body and while using the indicator 235 as a guide—and re-orient the short side 150 of the tubular portion 200 toward the new, subsequent area of concern. In this aspect, the indicator 235 informs the surgeon about the orientation of the tubular portion 200 without removing it from the incision 30, thereby reducing physical trauma to the tissue 10 near the incision 30. Once in the desired orientation, the surgeon can releasably re-secure the base portion 200 to the tissue 10 and then re-manipulate the surgical instrument 150 toward the subsequent area of concern. In this aspect, a number of different areas of concern near the same incision 30 can be reached with the surgical instrument 150 without removing the surgical port 100.

The surgeon may identify one or more additional places where treatment is desired which require a new incision. After releasing the sutures or other fastening means, the surgeon can remove the surgical port 100 and insert it into a subsequent opening, using the method steps described herein.

What is claimed is:

1. A surgical port comprising:
a hollow tubular portion defining a lumen extending therethrough along a central longitudinal axis from a proximal end to a distal end, wherein said tubular portion is beveled at an oblique angle with respect to said longitudinal axis to form a bevel tip that is sized and shaped to facilitate insertion of said tubular portion into an opening in a tissue; and
a base portion connected to said tubular portion and extending outwardly from said proximal end to a base edge, wherein said base portion is sized and shaped to inhibit insertion of said base portion into said opening, and wherein said base portion is sized and shaped to protect from injury said tissue near said opening.

2. The surgical port of claim 1, further comprising:
a fastening means for releasably securing said base portion to an outer surface of said tissue near said opening.

3. The surgical port of claim 2, wherein said fastening means comprises one or more holes through said base portion together with one or more sutures placed through said one or more holes.

4. The surgical port of claim 1, wherein said proximal end defines a generally circular proximal opening oriented along a plane generally perpendicular to said central longitudinal axis,
wherein said tubular portion comprises a long side extending from said base portion to said bevel tip and a short side extending from said base portion to a bevel heel, and
wherein said distal end defines a generally elliptical distal opening extending from near said bevel tip to near said bevel heel.

5. The surgical port of claim 4, further comprising a surgical instrument inserted through said lumen,
wherein said short side of said tubular portion facilitates lateral movement of said surgical instrument relative to said longitudinal axis.

6. The surgical port of claim 5, wherein said oblique angle is selected such that said short side of said tubular portion is sized and shaped to facilitate lateral movement of said surgical instrument toward said short side.

7. The surgical port of claim 1, wherein said oblique angle is selected such that said bevel tip facilitates insertion of said tubular portion through said opening, and such that said distal end pushes apart said opening during insertion of said tubular portion.

8. The surgical port of claim 1, wherein said lumen has a substantially uniform diameter.

9. The surgical port of claim 1, wherein said base portion comprises a distal surface facing said tissue and a generally opposing proximal surface, said port further comprising:
an indicator positioned to align longitudinally with said bevel tip wherein said indicator is sized, shaped, and located on said proximal surface of said base portion to provide at least a visual cue about the position of said bevel tip.

10. The surgical port of claim 9, wherein said indicator is a sign selected from the group consisting of a notch in said base edge of said base portion, a protuberance on said proximal surface, an indentation on said proximal surface, and indicia appearing on said proximal surface.

11. A method of protecting tissue from injury, comprising the steps of:
selecting a surgical port comprising a hollow tubular portion connected to a base portion, wherein said tubular portion defines a lumen extending therethrough along a central longitudinal axis from a proximal end to a distal end, wherein said tubular portion is beveled at an oblique angle with respect to said longitudinal axis to form a bevel tip that is sized and shaped to facilitate insertion of said tubular portion into an opening in a tissue, wherein said base portion extends outwardly from said proximal end to a base edge, and wherein said base portion is sized and shaped to protect from injury said tissue near said opening;
inserting said hollow tubular portion into an opening in a tissue such that said lumen extends into a body cavity;
releasably securing said base portion to said tissue; and
moving a surgical instrument in and through said lumen in the performance of a surgical operation.

12. The method of claim 11, wherein said step of releasably securing said base portion to said tissue further comprises:
passing one or more sutures through one or more holes in said base portion.

13. The method of claim 11, wherein said tubular portion comprises a long side extending from said base portion to said bevel tip and a short side extending from said base portion to a bevel heel, said method further comprising:
before said step of securing, orienting said short side of said tubular portion toward an area of concern within said body cavity to facilitate lateral movement of a surgical instrument toward said area of concern; and
manipulating said surgical instrument laterally toward said area of concern in the performance of said surgical operation.

14. The method of claim 13, wherein said base portion comprises an indicator positioned to align longitudinally with said long side and said bevel tip, and wherein said step of orienting further comprises:
aligning said surgical port such that said indicator is positioned away from said area of concern.

15. The method of claim 14, further comprising:
releasing said base portion from said tissue near said opening;
without removing said tubular portion from said opening in said tissue, and using said indicator as a guide, re-orienting said short side of said tubular portion toward a subsequent area of concern within said body cavity;
releasably re-securing said base portion to said tissue; and
re-manipulating said surgical instrument laterally toward said subsequent area of concern in the performance of said surgical operation.
